(19) United States
(12) Patent Application Publication
(10) Pub. No.: US 2026/0004926 A1
(43) Pub. Date: Jan. 1, 2026

(54) OPERATION CONTROL DEVICE, OPERATION CONTROL METHOD, AND COMPUTER PROGRAM PRODUCT

(71) Applicant: JVCKENWOOD Corporation, Yokohama-shi (JP)

(72) Inventor: Takamitsu Shimakura, Yokohama-shi (JP)

(21) Appl. No.: 19/321,369

(22) Filed: Sep. 8, 2025

Related U.S. Application Data
(63) Continuation of application No. PCT/JP2024/009024, filed on Mar. 8, 2024.

(30) Foreign Application Priority Data
Mar. 14, 2023 (JP) 2023-039290
Feb. 1, 2024 (JP) 2024-014105

(51) Int. Cl. G16H 40/60; A61B 5/00

(52) U.S. Cl. CPC G16H 40/60; A61B 5/4809

(57) ABSTRACT

An operation control device includes a brain information obtaining unit that obtains brain information of a user; a sleep determining unit that, based on the brain information obtained by the brain information obtaining unit, determines whether or not the user is in the sleep state; and a mode switching unit that sets a first-type mode in which, when the sleep determining unit determines that the user is in the awake state, the operation target is operated based on the brain information of the user, and sets a second-type mode in which, when the sleep determining unit determines that the user is in the sleep state, the operation target is operated based on the information other than the brain information of the user.

### CROSS-REFERENCE TO RELATED APPLICATIONS

This application is a Continuation of PCT International Application No. PCT/JP2024/009024 filed on Mar. 8, 2024 which claims the benefit of priority from Japanese Patent Application No. 2023-039290, filed on Mar. 14, 2023 and Japanese Patent Application No. 2024-014105, filed on Feb. 1, 2024, the entire contents of all of which are incorporated herein by reference.

### BACKGROUND OF THE INVENTION

### 1. Field of the Invention

The application concerned is related to an operation control device, an operation control method, and a computer program product.

### 2. Description of the Related Art

In recent years, there has been advancements in the technology for measuring brain activation information; and the technology of a brain-machine interface, which serves as an interface between the brain and the outside, is becoming feasible. In Japanese Patent Application Laid-open No. 2006-289565 mentioned below, the explanation is given about installing a first-type sensor for measuring the electric field generated accompanying the brain activity; installing a second-type sensor for detecting the state of the cerebral blood flow; and, based on the signals indicating the in-brain electric field obtained by the sensors, analyzing the brain activity of the operator and accordingly operating a robot.

However, in Japanese Patent Application Laid-open No. 2006-289565, there is no disclosure about the operation of a robot by a subject who is in the sleep state.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

An operation control device according to the present disclosure comprising: a brain information obtaining unit that obtains brain information of a user; a sleep determining unit that, based on the brain information obtained by the brain information obtaining unit, determines whether or not the user is in sleep state; and a mode switching unit that sets a first-type mode in which, when the sleep determining unit determines that the user is in awake state, an operation target is operated based on the brain information of the user, and a second-type mode in which, when the sleep determining unit determines that the user is in sleep state, an operation target is operated based on information other than the brain information of the user.

An operation control method according to the present disclosure comprising: obtaining brain information of a user; determining, based on the obtained brain information, whether or not the user is in sleep state; and
switching that includes setting a first-type mode in which, when it is determined that the user is in awake state, an operation target is operated based on the brain information of the user, and setting a second-type mode in which, when it is determined that the user is in sleep state, an operation target is operated based on information other than the brain information of the user.

A computer program product according to the present disclosure having a computer readable medium including a computer program, wherein the computer program, when executed by a computer, causes the computer to execute: obtaining brain information of a user; determining, based on the obtained brain information, whether or not the user is in sleep state; and
switching that includes setting a first-type mode in which, when it is determined that the user is in awake state, an operation target is operated based on the brain information of the user, and setting a second-type mode in which, when it is determined that the user is in sleep state, an operation target is operated based on information other than the brain information of the user.

### BRIEF DESCRIPTION OF THE DRAWINGS

FIG. 1 is a block configuration diagram illustrating an operation control device according to an embodiment;

FIG. 2 is a schematic diagram illustrating the details about the modes for switching set corresponding to the awake state and the sleep state by an operation control device;

FIG. 3 is a schematic diagram illustrating the details about the modes for switching set during the sleep state by the operation control device; and

FIG. 4 is flowchart for explaining an operation control method according to the present embodiment.

### DETAILED DESCRIPTION OF THE PREFERRED EMBODIMENTS

An exemplary embodiment of an operation control device, an operation control method, and a computer program product according to the application concerned is described below in detail with reference to the accompanying drawings. However, the present invention is not limited by the embodiment described below.

### Operation Control Device

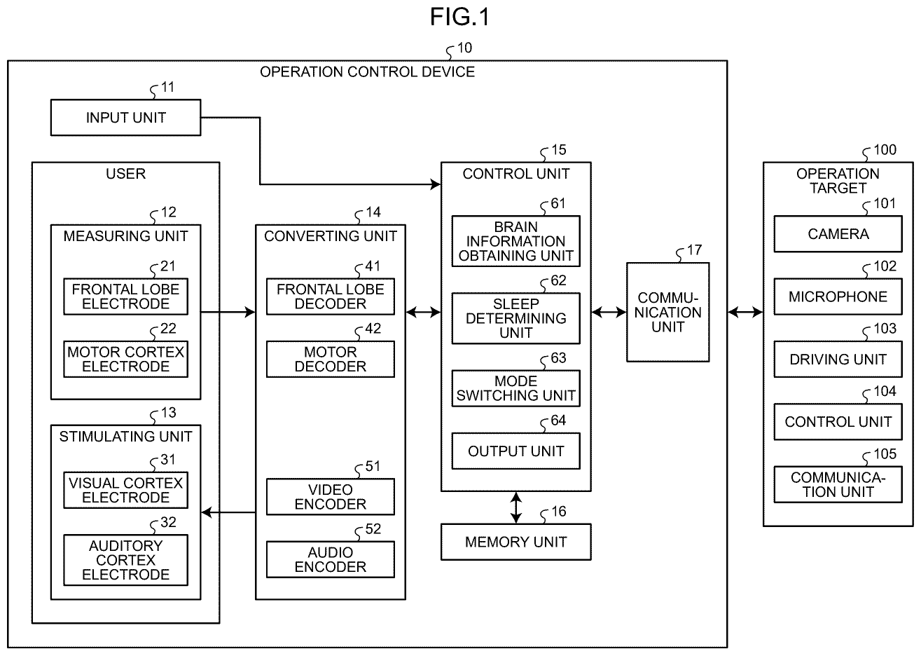

FIG. 1 is a block configuration diagram illustrating an operation control device according to the present embodiment.

As illustrated in FIG. 1, an operation control device 10 performs operation control of an operation target 100 based on the brain information of a user. The operation control device 10 includes an input unit 11, a measuring unit 12, a stimulating unit 13, a converting unit 14, a control unit 15, a memory unit 16, and a communication unit 17.

The input unit 11 is connected to the control unit 15. The input unit 11 is configured to be operable by a user and is capable of inputting various signals to the control unit 15. For example, to the control unit 15, the input unit 11 inputs a start signal for starting operation control meant for operating the operation target 100 based on the brain information, and inputs an end signal for ending the operation control meant for operating the operation target 100 based on the brain information. The input unit 11 can be implemented using, for example, a touch-sensitive panel, a button, a switch, or a keyboard.

The measuring unit 12 and the stimulating unit 13 are meant to be worn by the user. For example, the measuring unit 12 and the stimulating unit 13 are attached to the head region of the user. The measuring unit 12 includes a frontal lobe electrode 21 and a motor cortex electrode 22. The stimulating unit 13 includes a visual cortex electrode 31 and an auditory cortex electrode 32.

The measuring unit 12 obtains the brain waves representing the brain information of the user. For example, the measuring unit 12 includes an electrical sensor (for example, an electrode) that uses invasive electrodes and detects the brain waves coming out from the weak electrical current flowing through the neural network of the brain. When the user receives an external stimulation, the measuring unit 12 detects the electrical potential of the weak electrical current (i.e., detect electrical signals) based on the thoughts such as the mindset of the user.

The stimulating unit 13 provides stimulation by applying brain waves, which represent the brain information, to the user. For example, the stimulating unit 13 includes an electrical sensor (for example, an electrode) that applies the brain waves in the form of a weak electrical current to the neural network of the brain. The stimulating unit 13 applies the electrical potential of a weak electrical current (i.e., applies electrical signals) to the user based on the events occurring on the outside.

In the measuring unit 12, the frontal lobe electrode 21 is disposed at the position corresponding to the frontal lobe of the user. The frontal lobe electrode 21 obtains electrical signals of the brain waves representing the brain information corresponding to the frontal lobe of the user. The motor cortex is disposed at the position corresponding to the motor cortex in the brain of the user. The motor cortex electrode 22 obtains the brain waves representing the brain information corresponding to the motor cortex of the user.

In the stimulating unit 13, the visual cortex electrode 31 is disposed at the position corresponding to the visual cortex in the brain of the user. The visual cortex electrode 31 applies, as stimulation to the user, electrical signals of the brain waves representing the brain information corresponding to the visual cortex. The auditory cortex electrode 32 is disposed at the position corresponding to the auditory cortex in the brain of the user. The auditory cortex electrode 32 applies, as stimulation to the user, electrical signals of the brain waves representing the brain information corresponding to the auditory cortex.

The converting unit 14 includes a frontal lobe decoder 41, a motor decoder 42, a video encoder 51, and an audio encoder 52. The converting unit 14 can be configured to be an independent unit, or can be attached to the head region of the user in an integrated manner with the measuring unit 12 and the stimulating unit 13, or can be configured in an integrated manner with the control unit 15 but separately from the measuring unit 12 and the stimulating unit 13.

The frontal lobe decoder 41 is connected to the frontal lobe electrode 21 of the measuring unit 12. The frontal lobe decoder 41 restores the electrical signals of the brain waves of the user, which are input from the frontal lobe electrode 21, to the thought information of the user. The motor decoder 42 is connected to the motor cortex electrode 22 in the measuring unit 12. The motor decoder 42 restores the electrical signals of the brain waves of the user, which are input to the motor cortex electrode 22, to the thought information of the user. In that case, a plurality of electrical signals of the brain waves of the user is associated in advance with the thought information of the user. For example, using machine learning based on deep learning, the electrical signals of the brain waves are associated with the electrical signals of the brain waves with the thought information of the user.

The video encoder 51 is connected to the visual cortex electrode 31 in the stimulating unit 13. The video encoder 51 converts the thought information of the user into the electrical signals of the brain waves of the user, and outputs the electrical signals to the visual cortex electrode 31. The audio encoder 52 is connected to the auditory cortex electrode 32 in the stimulating unit 13. The audio encoder 52 converts the thought information of the user into the electrical signals of the brain waves, and outputs the electrical signals to the auditory cortex electrode 32. In that case, a plurality of electrical signals of the brain waves of the user is associated in advance with the thought information of the user. For example, using machine learning based on deep learning, the electrical signals of the brain waves are associated with the electrical signals of the brain waves with the thought information of the user.

The control unit 15 is connected to the input unit 11 and the converting unit 14. The control unit 15 receives a variety of information from the input unit 11 and the converting unit 14, as well as outputs a variety of information to the converting unit 14. The control unit 15 includes a brain information obtaining unit 61, a sleep determining unit 62, a mode switching unit 63, and an output unit 64. The control unit 15 is configured using, for example, an arithmetic circuit such as a central processing unit (CPU).

The brain information obtaining unit 61 obtains the brain information of the user. The brain information obtaining unit 61 obtains the electrical signals of the brain waves of the user as detected by the frontal lobe electrode 21 of the measuring unit 12, and obtains the thought information of the user as obtained by the frontal lobe decoder 41 by converting the electrical signals of the brain waves of the user. Moreover, the brain information obtaining unit 61 obtains the electrical signals of the brain waves of the user as detected by the motor cortex electrode 22 of the measuring unit 12, and obtains the thought information of the user as obtained by the motor decoder 42 by converting the electrical signals of the brain waves of the user.

The sleep determining unit 62 determines the sleep state of the user based on the brain information obtained by the brain information obtaining unit 61. In that case, based on the brain waves of the user as obtained by the frontal lobe electrode 21 and/or based on the brain waves of the user as obtained by the motor cortex electrode 22, the sleep determining unit 62 determines the sleep state of the user. More particularly, based on the brain information of the user, the sleep determining unit 62 determines whether the user is in the sleep state or in the awake state. When it is determined that the user is in the sleep state, based on the brain information of the user, the sleep determining unit 62 determines whether the user is in the REM sleep state or in the non-REM sleep state. When it is determined that the user is in the REM sleep state, based on the sleep information of the user, the sleep determining unit 62 determines whether or not the user is in the lucid dream state in which the degree of activity of the user is higher than a threshold value set in advance.

Herein, whether or not the user is in the sleep state is determined based on the brain waves of the user. For example, when the user is in the awake state, with reference to the reference value of the α waves having the dominant rhythm between 8 Hz and 13 Hz as the frequency component of the waveform, the detected brain waves of the user include the δ waves (between 0.5 Hz and 3.0 Hz) representing the slow waves having a lower frequency than the reference value and include the θ waves (between 4 Hz and 7.0 Hz). At that time, when the α waves as well as the δ waves have a higher frequency than a threshold value set in advance, it is determined that the user is in the sleep state. On the other hand, when at least either the α waves or the δ waves have a lower frequency than the threshold value, it is determined that the user is in the awake state.

The brain waves of the user have the waveform formed as a result of overlapping of a large number of waves having different frequency bands. When the user closes the eyes and goes to sleep, the visual information gets blocked and the eyesight-related component of the brain activity becomes weak. Subsequently, when the user gradually becomes unaware of the surrounding sounds, the hearing-related brain waves also become weak. As the user falls into a deep sleep, the constituent elements of the brain waves gradually become simplified. The type of sleep in which the user loses awareness and the brain waves become simplified is called the non-REM sleep that is divided into the following three stages: the first stage (non-REM sleep 1: N1) in which there is a decrease in the high-frequency α waves seen in the awake state and in which the low-frequency θ waves (between 4 Hz and 8 Hz) appear; the second stage (non-REM sleep 2: N2) in which the K complex waves appear or the sleep spindle appears; and the third stage in which the δ waves having a low frequency band (between 0.5 Hz and 2 Hz) go on increasing and account for 20% or more of the determination period (30 seconds).

On the other hand, in the REM sleep, although the body is sleeping, the brain remains active. The REM sleep is named after the initial letters “REM” of the term “rapid eye movement” in which the eyeballs move to the left and right beneath the closed eyelids and in which, although the muscles go in the most relaxed state, the brain remains partially active and dreams are seen often. During the REM sleep, it is believed that information processing such as consolidation of memory is carried out. Immediately after the user falls asleep, usually the non-REM sleep state is attained and there occurs transition from shallow non-REM sleep to deep non-REM sleep; and that is followed by the REM sleep state for a short duration. After that, the non-REM sleep state and the REM sleep state occur repeatedly in an alternate manner. For that reason, when the brain waves of the user are obtained and analyzed, it becomes possible to determine the non-REM sleep state and the REM sleep state.

Meanwhile, the REM sleep state involves subconscious simulation. On the other hand, lucid dreaming also represents simulation, but involves utilization of the brain in the conscious state of the user. During the non-REM sleep, the cerebral cortex of the user loses association with the other parts of the brain. Hence, it is believed that the dreams seen in that state are not much complex and are uninteresting. On the other hand, during the REM sleep, the cerebral cortex becomes active and starts establishing association with the other parts of the brain. When the user is having a lucid dream, the dorsolateral prefrontal cortex starts becoming active and it is believed that there is firm self-awareness and a story can be created by oneself. For that reason, when it is determined that the user is in the REM sleep state and when the degree of activity of the dorsolateral prefrontal cortex is higher than a threshold value, the sleep determining unit 62 determines that the user is in the lucid dream state.

The sleep determining unit 62 determines the sleep state and the awake state of the user based on the brain information obtained by the brain information obtaining unit 61. Herein, the brain information is not limited to the brain waves explained above, and the determination can be performed alternatively using, for example, the cerebral blood flow.

The mode switching unit 63 sets the mode based on the sleep state (the awake state) of the user as determined by the sleep determining unit 62. The modes for switching include an active mode (a first-type mode) and sleep modes (second-type modes). More particularly, the active mode, a lucid dream mode (the sleep mode), and an auto mode (the sleep mode) are set as the modes for switching.

When the sleep determining unit 62 determines that the user is not in the sleep state but is in the awake state, the mode switching unit 63 selects and sets the awake mode. The awake mode is, what is called, a brain information control mode in which the operation target 100 is operated based on the brain information belonging to the frontal lobe of the user.

When the sleep determining unit 62 determines that the user is in the sleep state, the mode switching unit 63 selects and sets a sleep mode, that is, selects and sets either the lucid dream mode or the auto mode. At that time, when it is determined that the user is in the REM sleep state and the degree of brain activity is higher than a threshold value set in advance, the mode switching unit 63 sets the lucid dream mode. In the lucid dream mode, the operation target 100 is operated based on the information belonging to the motor cortex of the user.

On the other hand, when the sleep determining unit 62 determines that the user is not in the REM sleep state, the mode switching unit 63 sets the auto mode. Moreover, even when the user is in the REM sleep state, if the degree of brain activity is equal to or lower than the threshold value, the mode switching unit 63 sets the auto mode. In the auto mode, the operation target 100 is operated based on a computer program set in advance.

Regarding the details about the awake mode, the lucid dream mode, and the auto mode; the explanation is given later.

The output unit 64 outputs an operation signal meant for operating the operation target 100 based on either the awake mode, or the lucid dream mode, or the auto mode as set by the mode switching unit 63. Moreover, the output unit 64 outputs video signals and audio signals, which are input from the operation target 100, to the video encoder 51 and the audio encoder 52 of the converting unit 14.

The memory unit 16 is used to store a computer program that the control unit 15 executes to perform operation control. The memory unit 16 is an external storage device such as a hard disk drive (HDD), or is a memory. The memory unit 16 is also used to store threshold values that the control unit 15 uses in performing various determination operations.

The communication unit 17 is connected to the control unit 15. The communication unit 105 is capable of sending a variety of information to and receiving a variety of information from the operation target 100 based on command signals received from the control unit 15.

Meanwhile, although not illustrated in FIG. 1, the control unit 15 can also include an eyeball detecting unit that detects the eye movement of the user. The eyeball detecting unit is implemented according to an optical method or an electrical method. More particularly, the known methods include the double Purkinje method, the scleral reflection method, the search coil method, and the electrooculogram method. For example, the cornea side of an eye is positively charged, and the retinal side is negatively charged. Thus, if an electrode is placed at the bridge of the nose, if an electrode is placed at the outer corner of an eye, and if the potential difference between the electrodes is measured; the electrode placed at the outer corner of the eye functions as the positive electrode, and the electrode placed at the bridge of the nose functions as the negative electrode. From the potential difference between the two electrodes, the angle of rotation of the eyeball can be calculated. Herein, the eye movement of the user is also included in the information about the motor cortex of the user.

Examples of the operation target 100 include, but are not limited to, a robot. Thus, any device that is operable based on communication can be used as the operation target 100. The operation target 100 can be connected to the operation control device 10. The operation target 100 includes a camera 101, a microphone 102, a driving unit 103, a control unit 104, and a communication unit 105. The camera 101 obtains videos of the surroundings of the operation target 100. The microphone 102 obtains sounds of the surroundings of the operation target 100. The driving unit 103 drives the operation target 100. When the operation target 100 has arms and legs, the driving unit 103 operates the arms and legs. When the operation target 100 is a vehicle or an airplane, the driving unit 103 represents a wheel, or crawler, or a rotor. The control unit 104 is connected to the camera 101, the microphone 102, the driving unit 103, and the communication unit 105; and is capable of controlling those constituent elements. The communication unit 105 is capable of sending a variety of information to and receiving a variety of information from the control unit 15 via the communication unit 17.

The control unit 104 outputs the videos, which are obtained by the camera 101, and the sounds, which are obtained by the microphone 102, to the operation control device 10 via the communication unit 105. Moreover, the control unit 104 performs drive control of the driving unit 103 based on a signal input from the operation control device 10 via the communication unit 105.

### Modes for Switching

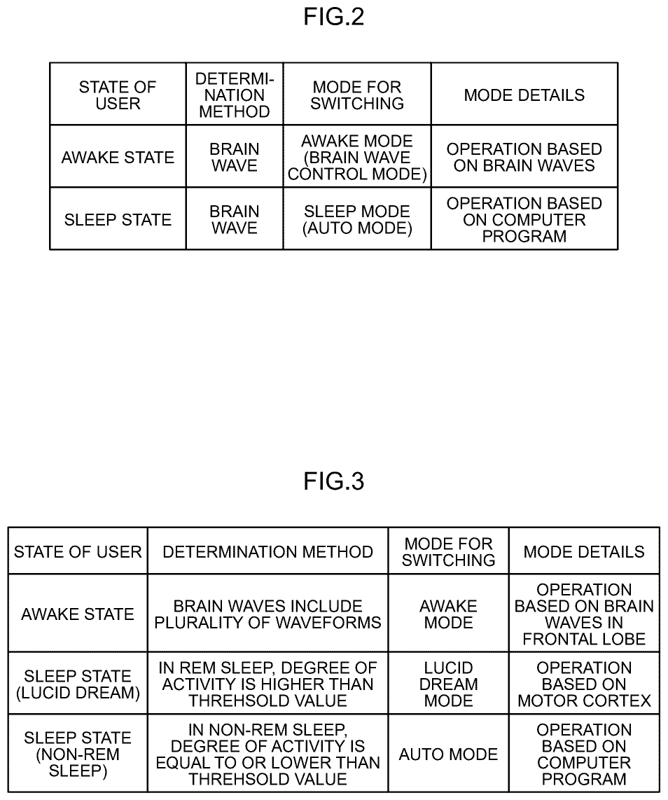

FIG. 2 is a schematic diagram illustrating the details about the modes for switching set corresponding to the awake state and the sleep state by the operation control device. FIG. 3 is a schematic diagram illustrating the details about the modes for switching set during the sleep state by the operation control device.

As illustrated in FIGS. 1 and 2, according to the sleep state (the awake state) of the user, the mode switching unit 63 selects and sets either the awake mode or the sleep mode. When the brain waves of the user include a plurality of waveforms, it is determined that the user is in the awake state, and the mode switching unit 63 sets the awake mode. The awake mode represents the brain wave control mode, and the output unit 64 operates the operation target 100 based on the brain information belonging to the frontal lobe of the user as obtained by the frontal lobe electrode 21 of the measuring unit 12 and converted by the frontal lobe decoder 41.

When the brain waves of the user do not include a plurality of waveforms, it is determined that the user is in the sleep state, and the mode switching unit 63 sets a sleep mode. The sleep modes include the auto mode, and the output unit 64 operates the operation target 100 based on the computer program stored in the memory unit 16. The memory unit 16 is connected to the control unit 15 (the mode switching unit 63). However, alternatively, the memory unit 16 can be connected to the control unit 104 of the operation target 100.

The operations performed by the mode switching unit 63 are not limited to the explanation given above. As illustrated in FIGS. 1 and 3, according to the sleep state (the awake state) of the user, the mode switching unit 63 selects and sets one mode from among the awake mode, the lucid dream mode (a sleep mode), and the auto mode (a sleep mode). When the brain waves of the user include a plurality of waveforms, it is determined that the user is in the awake state, and the mode switching unit 63 sets the awake mode. In the awake mode, the output unit 64 operates the operation target 100 based on the brain information belonging to the frontal lobe of the user as obtained by the frontal lobe electrode 21 of the measuring unit 12 and converted by the frontal lobe decoder 41.

When the user is in the REM sleep state and when the degree of activity is higher than a threshold value, the mode switching unit 63 determines that the user is in the lucid dream state, and sets the lucid dream mode. In the lucid dream mode, the output unit 64 operates the operation target 100 based on the information belonging to the motor cortex of the user as obtained by the motor cortex electrode 22 of the measuring unit 12 and converted by the motor decoder 42.

Alternatively, after the mode switching unit 63 has set the lucid dream mode, in the lucid dream mode, the output unit 64 can be configured to operate the operation target 100 based on the detection result of the eyeball detecting unit. That is, the output unit 64 operates the operation target 100 based on the eye movement of the user representing the information belonging to the motor cortex of the user as obtained by the eye detecting unit.

When the user is in the non-REM sleep state or when the user is in the REM sleep state but with the degree of activity to be equal to or lower than the threshold value, the mode switching unit 63 determines that the user is in the non-REM sleep state and sets the auto mode. In the auto mode, the output unit 64 operates the operation target 100 based on the computer program stored in the memory unit 16.

### Operation Control Method

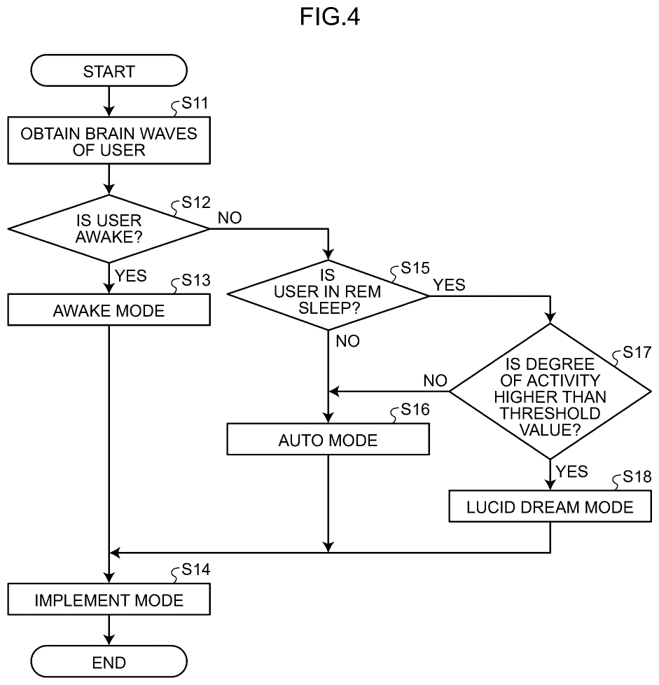

FIG. 4 is flowchart for explaining the operation control method according to the present embodiment.

As illustrated in FIGS. 1 and 4, at Step S11, the brain information obtaining unit 61 obtains, as the brain information of the user, the electrical signals of the brain waves of the user and the thought information of the user. At Step S12, based on the brain information of the user, the sleep determining unit 62 determines whether or not the user is in the sleep state (the awake state). When the sleep determining unit 62 determines that the user is not in the sleep state but is in the awake state (Yes), the system control proceeds to Step S13. At Step S13, since the sleep determining unit 62 has determined that the user is in the awake state, the mode switching unit 63 sets the awake mode (the brain wave control mode). At Step S14, the output unit 64 implements the brain wave control mode. That is, the output unit 64 operates the operation target 100 based on the brain information belonging to the frontal lobe of the user. For example, the output unit 64 continuously outputs the brain information of the user to the control unit 104 via the communication units 17 and 107. Then, the control unit 104 operates the operation target 100 based on the received brain information of the user.

Meanwhile, at Step S12, when the sleep determining unit 62 determines that the user is in the sleep state and not in the awake state (No), the system control proceeds to Step S15. At Step S15, the sleep determining unit 62 determines whether or not the user is in the REM sleep state. When the sleep determining unit 62 determines that the user is not in the REM sleep state (No), the system control proceeds to Step S16. At Step S16, since the sleep determining unit 62 has determined that the user is not in the REM sleep state, that is, has determined that the user is in the non-REM sleep state, the mode switching unit 63 sets the auto mode that is one of the sleep modes. At Step S14, the output unit 64 implements the auto mode. That is, the output unit 64 operates the operation target 100 based on a computer program set in advance. For example, the output unit 64 outputs an auto-mode computer program, which is stored in the memory unit 16, to the control unit 104 via the communication units 17 and 107. Then, the control unit 104 operates the operation target 100 based on the received auto-mode computer program. After sending the auto-mode computer program, the output unit 64 can cut off the communication happening via the communication units 17 and 107.

Meanwhile, at Step S15, when the sleep determining unit 62 determines that the user is in the REM sleep state (Yes), the system control proceeds to Step S17. At Step S17, the sleep determining unit 62 determines whether or not the degree of brain activity of the user is higher than a threshold value. When the sleep determining unit 62 determines that the degree of brain activity of the user is equal to or lower than the threshold value (No), the system control proceeds to Step S16 and an identical operation is performed to the operation explained earlier. On the other hand, when the sleep determining unit 62 determines that the degree of brain activity of the user is higher than the threshold value (Yes), the system control proceeds to Step S18. At Step S18, since the sleep determining unit 62 has determined that the user is in the lucid sleep state in which the degree of brain activity is higher than the threshold value, the mode switching unit 63 sets the lucid dream mode that is one of the sleep modes. Thus, at Step S14, the output unit 64 implements the lucid dream mode. That is, the output unit 64 operates the operation target 100 based on the information belonging to the motor cortex of the user. For example, the output unit 64 continuously outputs the information belonging to the motor cortex of the user to the control unit 104 via the communication units 17 and 107. Then, the control unit 104 operates the operation target 100 based on the received information belonging to the motor cortex of the user.

In the embodiment described above, the stimulating unit 13 includes the visual cortex electrode 31 and the auditory cortex electrode 32. However, that is not the only possible case. For example, the visual cortex electrode 31 and the video encoder 51 can be replaced with a device that projects videos onto the retinas from the contact lenses being worn by the user. Moreover, the auditory cortex electrode 32 and the audio encoder 52 can be replaced with a device that reproduces sounds from the headphones being worn by the user.

Moreover, in the embodiment described above, the visual cortex electrode 31 inputs the brain waves to the visual cortex of the user. Alternatively, the visual cortex electrode 31 can input the brain waves to the entire brain of the user. Similarly, the auditory cortex electrode 32 inputs the brain waves to the auditory cortex of the user. Alternatively, the auditory cortex electrode 32 can input the brain waves to the entire brain of the user.

Furthermore, in the embodiment described above, the motor cortex electrode 22 obtains the brain waves from the motor cortex of the user. Alternatively, the motor cortex electrode 22 can obtain the brain waves from the entire brain. Moreover, the motor cortex electrode 22 obtains the brain waves from the motor cortex of the user, and the motor decoder 42 decodes the brain waves and outputs them to the operation target 100. Alternatively, the operation target 100 can include a speaker, the motor decoder 42 can decode the brain waves of the user into sounds, and the sounds can be reproduced from the operation target 100.

Furthermore, in the embodiment described above, the sleep determining unit 62 determines the sleep state of the user based on the brain information of the user. Alternatively, the sleep state of the user can be determined when there occurs a decline in the correlation between the motor cortex signals obtained from the motor cortex electrode 22 and myoelectric signals obtained from an electromyography electrode (not illustrated).

Moreover, in the embodiment described above, when the sleep state of the user is determined based on the brain information of the user, specific stimulation can be applied to the user for the purpose of notifying that a dream is being seen; and the dream can be transitioned to a lucid dream so that the user can be prompted to control the dream.

Meanwhile, the output unit 64 outputs an operation signal for enabling operation of the operation target 100 based on each mode. That operation can be carried out in a periodic manner (for example, after every 0.01 seconds). Moreover, the output unit 64 outputs the video signals and the audio signals, which are input from the operation target 100, to the video encoder 51 and the audio encoder 52 of the converting unit 14. That operation too can be carried out in a periodic manner (for example, after every 0.01 seconds).

### Effects of Embodiment

The operation control device according to the present embodiment includes: the brain information obtaining unit 61 that obtains the brain information of the user; the sleep determining unit 62 that, based on the brain information obtained by the brain information obtaining unit 61, determines whether or not the user is in the sleep state; and the mode switching unit 63 that switches the mode between a first-type mode, in which the operation target 100 is operated based on the brain information of the user when the sleep determining unit 62 determines that the user is in the awake state, and a second-type mode, in which the operation target 100 is operated based on the information other than the brain information of the user when the sleep determining unit 62 determines that the user is in the sleep state.

Hence, the operation target 100 is operated based on the appropriate information depending on the awake state or the sleep state of the user; and, even when the user is in the sleep state, the operation target 100 can be operated by making use of the sleeping hours.

In the operation control device according to the present embodiment, the second-type modes include the auto mode in which the operation target is operated based on a computer program set in advance. Hence, when the user is in the sleep state, the auto mode is set and the operation target 100 is operated based on the computer program set in advance. Thus, even when the user is in the sleep state, the operation target 100 can be operated by making use of the sleeping hours.

The operation control device according to the present embodiment includes the memory unit 16 in which the computer program for implementation of the auto mode is stored. The memory unit 16 is connected to the mode switching unit 63 or the operation target 100. Hence, the auto mode can be implemented in a prompt manner.

The operation control device according to the present embodiment includes the output unit 64 that outputs an operation signal to the operation target 100 based on the sleep mode or the auto mode set by the mode switching unit 63. When the sleep mode is to be implemented, the output unit 64 enables outputting an operation signal to the operation target 100. When the auto mode is to be implemented, the output unit 64 blocks the output of an operation signal to the operation target 100. As a result, it becomes possible to simplify the operations with respect to the operation target 100.

For example, the sleep modes include the auto mode, and the output unit 64 operates the operation target 100 based on the computer program stored in the memory unit 16. However, that is not the only possible case. Thus, during a sleep mode, the output unit 64 can operate the operation target 100 for outputting an operation signal for reducing the power consumption of the operation target 100 or an operation signal for stopping the operation target. Moreover, during a sleep mode, the output unit 64 can output a signal that specifies another operation control device. When a signal specifying another operation control device is input to the operation target 100, the communication unit 105 can send a variety of information to and receive a variety of information from the control unit of the other operation control device via the communication unit of the other operation control device.

In the operation control device according to the present embodiment, the second-type modes include a sleep mode (the lucid dream mode) in which the operation target is operated based on the information belonging to the motor cortex of the user. Hence, when the user is in the sleep state, a sleep mode is set, and the operation target 100 is operated based on the information belonging the motor cortex of the user. Thus, even when the user is in the sleep state, the brain of the user and the operation target 100 are connected, and the operation of the operation target 100 can be carried out by making use of the sleeping hours and without hindering the brain activity in the active state of the user.

In the operation control device according to the present embodiment, when the sleep determining unit 62 determines that the user is in the sleep state and that the brain activity is higher than a threshold value set in advance, the mode switching unit 63 sets a sleep mode (the lucid dream mode). On the other hand, when the sleep determining unit 62 determines that the user is in the sleep state and that the brain activity is equal to or lower than the threshold value set in advance, the mode switching unit 63 sets the auto mode in which the operation target 100 is operated based on a computer program set in advance. Based on the sleep mode or the auto mode set by the mode switching unit 63, the output unit 64 outputs an operation signal to the operation target 100. Thus, the lucid dream mode is set when the degree of brain activity of the user is higher than a threshold value; and the auto mode is set when the degree of brain activity of the user is equal to or lower than the threshold value and the operation target 100 is operated based on a computer program. As a result, the operation target 100 can be appropriately operated during the sleep state of the user.

In the operation control device according to the present embodiment, when the sleep determining unit 62 determines that the user is not in the sleep state, the mode switching unit 63 sets the awake mode in which the operation target 100 is operated based on the brain information belonging to the frontal lobe of the user. Then, based on the awake mode set by the mode switching unit 63, the output unit 64 outputs an operation signal to the operation target 100. Thus, when the user is not in the sleep state, the awake mode is set and the operation target 100 is operated based on the brain information belonging to the frontal lobe of the user. As a result, the operation target 100 can be appropriately operated during the awake state of the user.

Till now, the explanation was given about the operation control device according to the application concerned. However, the application concerned can be implemented according to various other forms other than the embodiment described above.

The constituent elements of the operation control device illustrated in the drawings are merely conceptual, and need not be physically configured as illustrated. The constituent elements, as a whole or in part, can be separated or integrated either functionally or physically based on various types of loads or use conditions.

The operation control device is configured using, for example, a computer program that is loaded as software in a memory. In the embodiment described above, the configuration is explained with reference to function blocks implemented as a result of coordination between hardware and software. Such function blocks can be implemented in various ways, such as using only hardware, or using only software, or using a combination of hardware and software.

Meanwhile, the constituent elements described above are not to be thus limited but are to be construed as embodying all modifications and alternative constructions that may occur to one skilled in the art that fairly fall within the basic teaching herein set forth.

The information processing device, the information processing method, and the computer program product according to the application concerned can be implemented in a technology for controlling the brain activity of the user.

According to the application concerned, even when the user is asleep, the operation target can be made operable.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An operation control device comprising:
a brain information obtaining unit that obtains brain information of a user;
a sleep determining unit that, based on the brain information obtained by the brain information obtaining unit, determines whether or not the user is in sleep state; and
a mode switching unit that sets
a first-type mode in which, when the sleep determining unit determines that the user is in awake state, an operation target is operated based on the brain information of the user, and
a second-type mode in which, when the sleep determining unit determines that the user is in sleep state, an operation target is operated based on information other than the brain information of the user.

2. The operation control device according to claim 1, wherein the second-type mode includes an auto mode in which the operation target is operated based a computer program set in advance.

3. The operation control device according to claim 2, further comprising a memory unit in which a computer program to be executed during the second-type mode is stored, wherein
the memory unit is connected to the mode switching unit or the operation target.

4. The operation control device according to claim 1, further comprising an output unit that outputs an operation signal to the operation target based on the first-type mode or the second-type mode set by the mode switching unit, wherein
when the first-type mode is to be implemented, the output unit enables outputting an operation signal to the operation target, and
when the second-type mode is to be implemented, the output unit blocks output of an operation signal to the operation target.

5. The operation control device according to claim 1, wherein the second-type mode includes a sleep mode in which the operation target is operated based on information belonging to motor cortex of the user.

6. The operation control device according to claim 5, wherein
when the sleep determining unit determines that the user is in sleep state and that degree of activity is higher than a threshold value set in advance, the mode switching unit sets the sleep mode, and
when the sleep determining unit determines that the user is in sleep state and that degree of activity is equal to or lower than a threshold value set in advance, the mode switching unit sets the auto mode.

7. The operation control device according to claim 5, wherein, when the sleep determining unit determines that the user is in awake state, the mode switching unit sets an awake mode in which the operation target is operated based on brain information belonging to frontal lobe of the user.

8. An operation control method comprising:
obtaining brain information of a user;
determining, based on the obtained brain information, whether or not the user is in sleep state; and
switching that includes
setting a first-type mode in which, when it is determined that the user is in awake state, an operation target is operated based on the brain information of the user, and
setting a second-type mode in which, when it is determined that the user is in sleep state, an operation target is operated based on information other than the brain information of the user.

9. A computer program product having a computer readable medium including a computer program, wherein the computer program, when executed by a computer, causes the computer to execute:
obtaining brain information of a user;
determining, based on the obtained brain information, whether or not the user is in sleep state; and
switching that includes
setting a first-type mode in which, when it is determined that the user is in awake state, an operation target is operated based on the brain information of the user, and
setting a second-type mode in which, when it is determined that the user is in sleep state, an operation target is operated based on information other than the brain information of the user.
